(19) United States
(12) Patent Application Publication
(10) Pub. No.: US 2025/0104866 A1
(43) Pub. Date: Mar. 27, 2025

(54) SYSTEM AND METHOD FOR DIAGNOSING INFLAMMATORY BOWEL DISEASE STATE AND SEVERITY

(71) Applicant: The Cleveland Clinic Foundation, Cleveland, OH (US)

(72) Inventors: Thaddeus Stappenbeck, St. Louis, MO (US); Scott Tedmund Espenschied, Cleveland, OH (US); Daniel Shenfeld, Cleveland, OH (US); Michael Shafir, Cambridge, MA (US)

(21) Appl. No.: 18/891,486

(22) Filed: Sep. 20, 2024

Related U.S. Application Data
(60) Provisional application No. 63/539,832, filed on Sep. 22, 2023.

(51) Int. Cl. G16H 50/20; G06T 7/00; G06T 7/10; G16H 30/40

(52) U.S. Cl. CPC G16H 50/20; G06T 7/0012; G06T 7/10; G16H 30/40; G06T 2207/20081; G06T 2207/30024

(57) ABSTRACT

A method for determining a pathological parameter (e.g., a microvilli length (MVL)) of a subject is used to determine a therapeutic response for treatment of the subject. The method includes receiving a pathology image of the subject, identifying a pathological feature and determining a parameter of that feature (e.g., MVL), and determining an aggregate parameter. The pathology image may be pre-processed and/or segmented into regions of interest. The entire pathology image and/or the segmented regions may be subject to feature annotation.

### CROSS-REFERENCE TO RELATED APPLICATIONS

The present application claims the benefit of U.S. Provisional Patent Application No. 63/539,832 filed on Sep. 22, 2023, the entirety of which is incorporated by reference herein.

### BACKGROUND

The interior lumen of the small intestine is lined with infoldings of epithelial tissue called villi. The surface of the colon is flat. Nutrients are absorbed in the small intestine through this epithelial tissue; water is primarily absorbed in the colon also through epithelial tissue. Villi project from the folds of the tissue into the lumen, and are lined with epithelial cells; flat surface cuffs are present in the colon. Similarly, microvilli form microscopic fingerlike projections from the apical surface of epithelial cells into the lumen of the small intestine and colon. Each epithelial cell has ˜3,000 microvilli, and each microvillus is about 1-2 μm long and about 0.1 μm in diameter.

The microvilli are involved in a wide variety of functions, including absorption, secretion, cellular adhesion, and mechanotransduction. Particularly, the villi and microvilli effectively serve to increase the absorptive surface area of the lumen wall of the small intestine (10-fold and 100-fold, respectively), and help absorption of nutrients by the epithelial tissue. Determination of microvillus length (MVL) is helpful in determining its relationship with a therapeutic response for treatment of a type of inflammatory bowel disease, such as Crohn's disease or ulcerative colitis. For example, such a determination may directly influence a determination of absorptivity relative to a norm or reference, which may impact the therapeutic response.

Current techniques for measuring MVL require a clinician to manually analyze one or more patches of intestinal tissue on a pathology slide of a subject. These slides may be, for example, hematoxylin and eosin (H&E) stained samples of biopsy specimens captured using a standard laboratory transmitted light microscope typically using a color camera and 100× oil immersion objective lenses. More specifically, the clinician must first identify regions of the pathology slide that are suitable for analysis and then manually quantify the MVL. The quantification requires manually drawing a series of perpendicular lines across the brush border of epithelial cells, and measuring the length of each line.

Such manual analysis is subject to human error and imprecisions. Moreover, the clinician is limited in the number of slides that may be analyzed, the number of MVL measurements that may be determined in each slide, and the types of properties of MVL. For example, given the low throughput and tediousness of the method, manual quantification is traditionally performed on only 5 cells per villus, and 10 villi per sample (50 total cells per sample). These limitations are in part due to the resolution of the slides and human ability to discern differences thereof.

### BRIEF SUMMARY

According to one example of the present disclosure, a method comprises: receiving a digitized pathology image of a subject; segmenting the image to identify a region of interest that includes at least a portion of at least one villus; identifying a pathological feature of the image within the region of interest; determine a parameter pertaining to each of at least one instance of the identified pathological feature in the image; determining an aggregate parameter based on the determined parameter; and outputting a report of the determined aggregate parameter.

In various embodiments of the above example, the pathological feature is a microvillus; the pathological feature is a cell nucleus; the pathological feature is a goblet cell; the parameter is a microvillus length; the identified pathological feature is a villus, identifying at least one pathological feature comprises identifying contours corresponding to inner and outer edges of a brush border of at least one villi in the region of interest, determining the parameter comprises determining a distance between the contours, and the parameter is the distance between the contours; the identified pathological feature is a villus, identifying at least one pathological feature comprises identifying a contour corresponding to an edge of at least one villi in the region of interest and segmenting the contour, determining the parameter comprises, for each contour segment, determining a direction toward an intracellular compartment of an epithelial cell corresponding to the segment and determining a distance between the segment and a pixel having a drop in intensity greater than a threshold along a path that extends normal to the segment in the determined direction, and the parameter corresponds to the determined distances; the threshold is based on an average greatest pixel intensity drop across all of the segments; the method further comprises classifying or predicting a state or a degree of a disease of the subject based on the aggregate parameter; the method further comprises quantifying an intestinal health of the subject based on the aggregate parameter; the method further comprises identifying a treatment protocol for the subject based on the aggregate parameter; the method further comprises treating the subject based on the treatment protocol; the outputted report comprises a heatmap of the determined parameter or the determined aggregate parameter overlaid on the digitized image; the method further comprises comparing the determined parameter or the determined aggregate parameter across a plurality of regions of interest or a plurality of digitized images; and/or the aggregate parameter is determined based on a comparison of the determined parameter across a plurality of the identified pathological features, a plurality of regions of interest, or a plurality of digitized images.

According to another example of the present disclosure, a system comprises at least one memory and processor configured to execute the above example method and various embodiments. In some embodiments, the at least one processor comprises at least one trained machine learning system.

### BRIEF DESCRIPTION OF THE DRAWINGS

FIG. 1 illustrates a sample image at varying magnifications.

FIG. 2 illustrates an example system of the present disclosure.

FIG. 3 illustrates an example method of the present disclosure.

FIG. 4 illustrates a villi annotated according to the present disclosure.

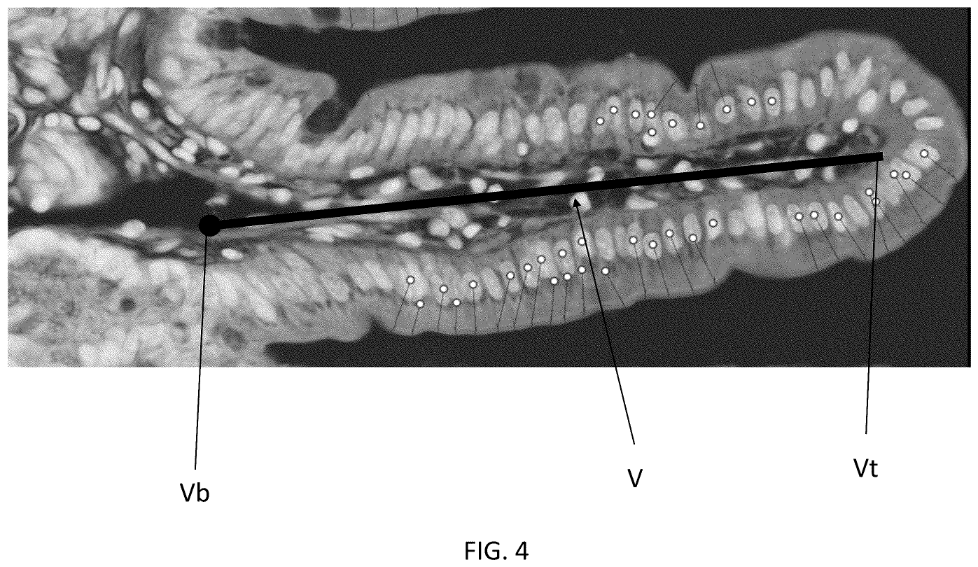

FIG. 5 illustrates a graph of an average MVL as a function of position along a villi.

FIG. 6 illustrates an example output of the method of the present disclosure.

### DETAILED DESCRIPTION

In view of the above, the system and method described herein relate to determining microvillus length or properties related to microvilli length of a subject. These determinations may be made, for example, from image processing and machine learning techniques of pathology samples of the subject. Further, in some embodiments, a diagnosis of inflammatory bowel disease type and severity may be determined and/or an appropriate determination and administration of a therapeutically effective treatment may be performed. Such MVL quantification can enable the capture and analysis of thousands of measurements per sample across multiple anatomical scales (cell, villus, anatomic region), and thus enable substantially deeper analysis of anatomical variance and microanatomical variance in MVL.

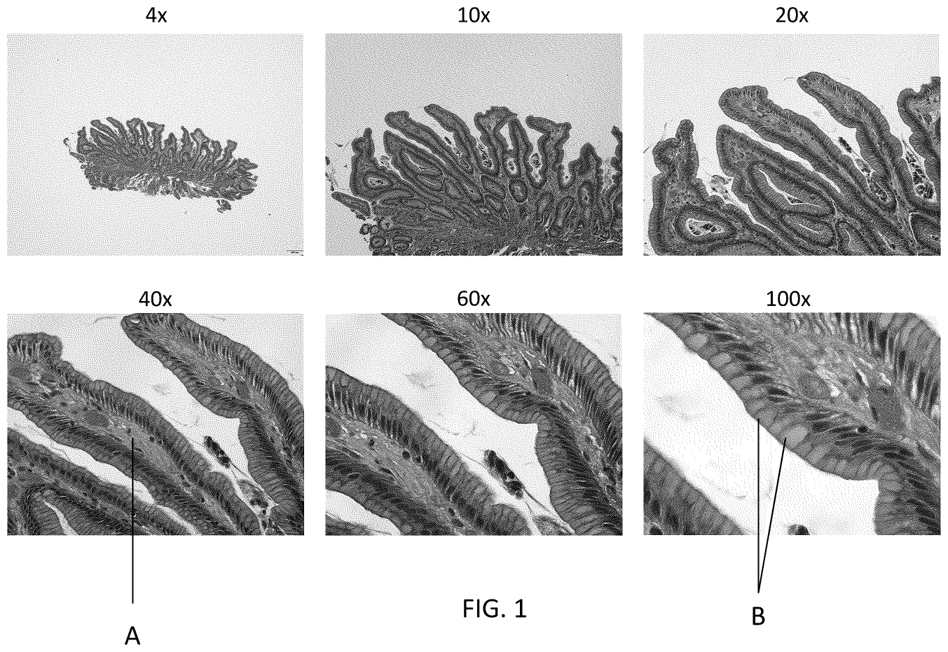

According to the present disclosure, a system comprising at least one memory and processor is configured to at least receive a digitized image, process the image to determine MVL of the image or a property related to MVL, and output the MVL, determined property, and/or diagnosis and/or treatment related to the MVL and/or property. The image may be one that is acquired by a microscope, or like medical imaging device, and be of a sample taken from a subject. In some examples, the sample may be an H&E stained pathology biopsy sample from the small intestine of the subject. Alternatively, the sample may be a sample from any other region of the gastrointestinal tract, including, but not limited to, the colon. As illustrated in FIG. 1, the image may depict any number of villi A that contain microvilli-bearing epithelial cells B of the sample, such as that belonging to a gut or intestine portion of the subject.

In addition, each image may be associated with metadata. The metadata may relate to the image itself and include, but is not limited to, when the image was taken, the type of instrument used to capture the image, the settings of said instrument, the location where the image was taken (e.g., anatomical, GPS, or the like), and the like. In addition, or alternatively, the metadata may include information about the subject of the image and include, but not limited to, age, gender, prior health information, and the like.

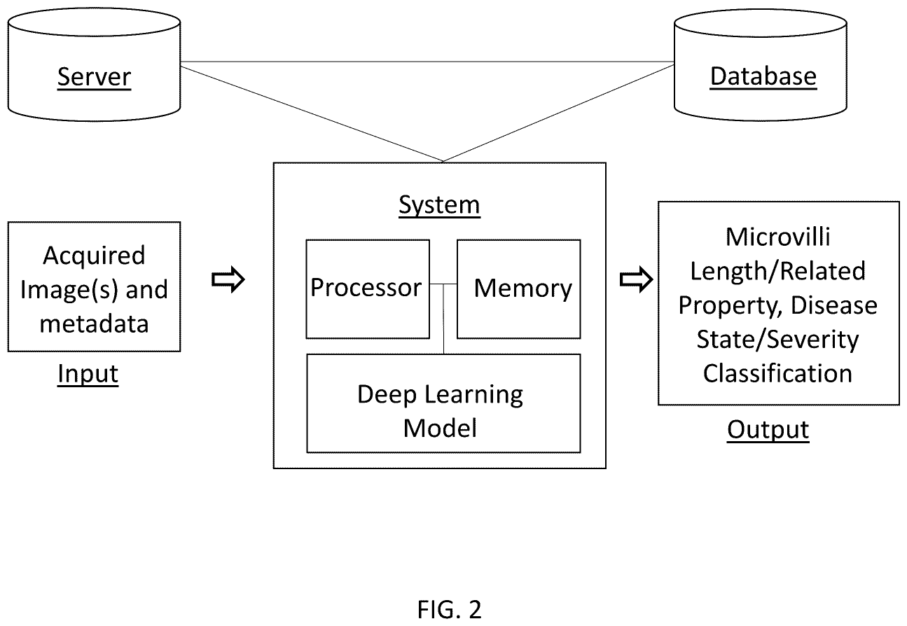

As illustrated in FIG. 2, the acquired image(s) and associated metadata may be received by the system via an upload from a server, a scan, a download, or directly from a camera. The images may be stored locally in a memory, or remotely, for example, in a remote database that may be in communication with the processor. The memory and/or the remote database may store a single image (and optionally associated metadata) or a plurality of images (and optionally associated metadata) that are collected over a period time. The processor receives and processes said image(s) via manual input by a user, as discussed above, an edge detection procedure, discussed below, and/or a deep learning model, also discussed below. After the image is processed, the corresponding output values are delivered to the user and/or stored in the memory or uploaded to the server or database.

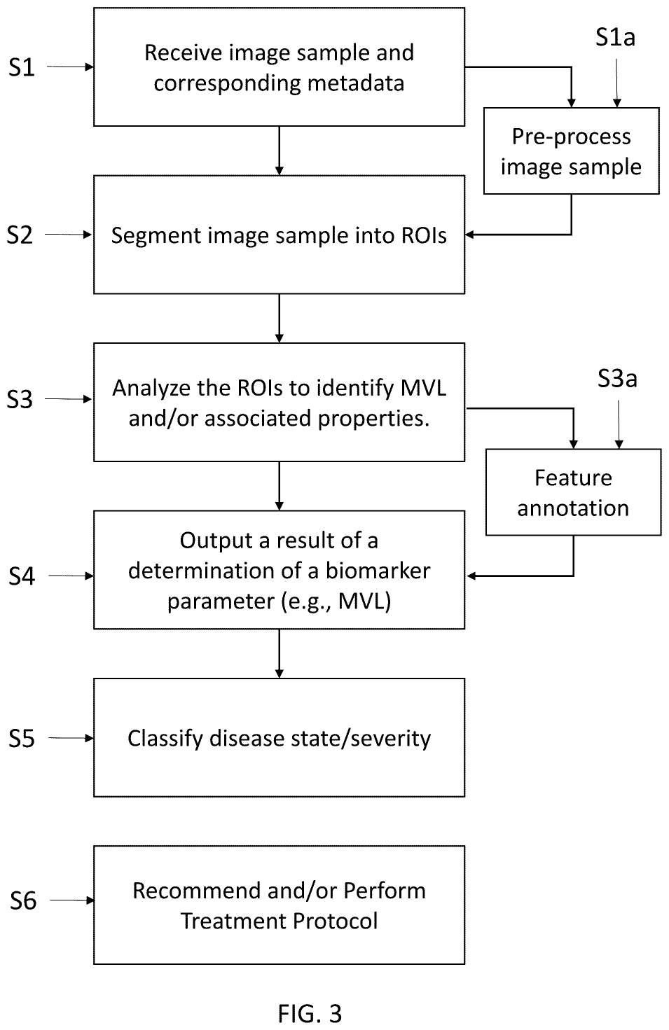

Briefly, a method of the present disclosure is illustrated in FIG. 3. As seen therein, the method of the present disclosure begins when an image sample and its corresponding metadata is received at step S1. As described above, the image may depict any number of villi that contain microvilli-bearing epithelial cells of the sample, while the metadata includes information about the image itself and/or the patient from which the sample came. At step S1a, the image sample is optionally pre-processed.

At step S2, the processor is configured to segment the image sample into one or more regions of interest (ROIs) based on how much of the captured epithelium is suitable for MVL quantification. Step S3 includes analyzing the segmented image sample to identify MVL and/or associated properties. Each selected ROI is selected for feature annotation at step S3a. This includes utilizing an annotation software in view of the general selection criteria.

The determined MVL and/or the property (e.g., a biomarker) related to MVL is then output to the user at step S4. Using the output values and/or properties, a user then is able to quantify a health of the tissue and therefore classify the disease state and/or severity at step S5. Finally at step S6, a treatment protocol may be determined and provided to a clinician, and/or a patient actually treated according to such a protocol, based on the identified disease state and/or severity.

More particularly, pre-processing may include, but is not limited to, brightness adjustment, contrast adjustment, conversion from RBG color to 8-bit grayscale, inversion of grayscale images, noise removal, filtering, normalization, and the like. In some embodiments, the images may be subject to a 40× or like magnification.

Following any pre-processing, the image may be segmented into one or more regions of interest (ROIs), such as those regions associated with villi. The segmented area may vary and can be determined by the user based on how much of the captured epithelium is suitable for MVL quantification. In one example, the one or more regions may include one or more whole villi, or regions of one or more villi. In one example, the regions may include a range of a lower third to a lower half of one or more villi, and a range of an upper third to an upper half of one or more villi. Segmentation may not be proportional to the size of the image, nor to the area of each biopsies in each scan.

The ROI may additionally or alternatively be identified to various selection criteria based on image processing and analysis. For example, such criteria may include whether the villi are intact and/or properly oriented, whether a brush border is in the same focal plane as the epithelial nuclei, whether the nuclei have typical basolateral positions, whether the region is free of debris and other histopathological processing artifacts, whether the anatomic region can be verified (i.e., does the biopsy have distinct ileal features and not colonic), and/or whether there is a suitable number of intact villi and cells for quantification.

Following segmentation, the segmented one or more regions of the image is then analyzed via image processing to identify MVL or associated properties of the regions of villi, discussed further below. For example, the analysis may include determining an MVL for one or more villi in the segmented region. Where the segments include a whole villus, the MVL may be determined along the total length of the villus; and where the segments include only a portion of a villus, the MVL may be determined as a length of that portion rather than a total length of the villus. In some embodiments, a thickness of the brush border may be determined as a proxy for MVL. This determination may be made by determining the total number of pixels along a path length and converting that number of pixels to a length based on a resolution of the image.

Each selected ROI can be selected for feature annotation. Feature annotation includes but is not limited to MVL, nuclei, villi, and goblet cells. Said feature annotation may be located in one annotation layer or multiple annotation layers. Multiple annotations may be enabled through layering, which allows for spatial pairing of different features. For example, an additional annotation layer is configured as a matching layer to pair MVL measurements with corresponding epithelial nuclei. In another example, following the annotation of microvilli, the nuclei may be annotated to pair MVL to a nucleus, which enables either multiple measurements per cell or exclusion of duplicate measurements. Subsequently, the villi may be annotated, allowing for further analysis, including but not limited to absolute and proportional enumeration of the number of annotated cells for a particular villus, and analysis of MVL along a villus axis.

Alternatively, or in addition to the above-described annotation of a selected ROI, an entire slide or image may be input for subsequent annotation. Specifically, an annotation software is configured to preview the entire slide or image and identify a region for MVL quantification, and/or other biomarkers, using the same general selection criteria described above. Other biomarkers relate to the quantification of a specific subset of intestinal epithelial cells (e.g., goblet cells). For example, the absolute and relative proportional abundance, area, average, pixel intensity, and variance in pixel intensity may be measured. In a further example, quantification of apoptotic bodies in the epithelial layer can be used as a proxy for cell death.

The intestinal epithelial brush border, comprised of microvilli, functions to increase the surface of the intestine to maximize absorption of ingested nutrients. In addition to passive transport of soluble, membrane permeable particles, there are numerous plasma membrane bound digestive enzymes and active transporters that further augment nutrient uptake. The thickness of the brush border is a proxy for MVL and can be determined using machine vision processes. For example, an edge detection technique (e.g., based on pixel intensity thresholding) may be used to identify boundaries of a line segment (i.e., the thickness of the brush border). Specifically, pixel intensity thresholding is used to sense where a microvillus begins and ends, and then the length of the line therebetween is measured.

For example, in a first method, an automated edge detection technique is performed or a user selects an appropriate location to be measured and draws a single line segment across the width of the brush border. This method is achieved by first identifying contours around brush border (e.g., the inner and outer edges of the brush border), for example, by using Canny edge detection technique. The two contours which are a furthest distance from the centroid of the selected area are then identified, and the nearest point on each contour is selected. Finally, a line segment between the two selected points is identified and length thereof determined.

A second method is achieved by first using an active contour technique to identify the outer border of the villus between two selected points. A middle portion (e.g., between 5 and 95% of the length) of the contour line may then be split into sections. The sections may be of any length, and may be of equal or non-equal length. In one example, the sections are divided by every 20 pixels. An intracellular compartment of the epithelial cell may then be determined, for example, by identifying a side of the contour line segment having a higher pixel intensity (i.e., that is brighter). In some embodiments, this determination may be made based on pixels along a path extending from a centroid of, and normal to, the contour line segment in both directions. This determination is preferably made for each segment with the path extending into the intracellular space up to a length of, for example, 50 pixels prior to being clipped at a length corresponding to the largest drop in pixel intensity averaged across all segments. Additionally, a quality control step may be performed in which lines are removed from the selected contour if the pixel intensity along the length of the line(s) are too low overall or if there is an insufficient change in pixel intensity. The boundaries (i.e., the beginning and end) of villi may be determined manually by a user, or automated by applying a segmentation technique or a machine learning system (e.g., including a convolutional neural network). This second method also allows for assessment of intracellular heterogeneity of MVL.

Further, aggregate properties related to the determined MVL may be determined for one or more of the regions and images. For example, any statistical representation of determined MVL may be made. These representations may include an average MVL, a maximum MVL, a minimum MVL, a median MVL, MVL variance, or the like, for one or more regions and/or images. In addition, the processor may be configured to analyze a slope, an area, anatomical coordinates, a location, a height, a shape, and/or any combination thereof, of any number of microvilli. Further, more involved measurements such as the curvature of microvillar brush, multidimensional splines and/or Gaussian Process models may be used.

These parameters may be determined using standard algebraic and statistical approaches. In some embodiments, the above parameters may be determined using SQL database queries to databases that associate MVL properties with the various parameters. Some parameters may be determined post hoc using statistical analysis computing software. By way of example, the processor may be configured to determine a decrease in slope from one surface point to a second surface point relative to a region of the villus.

In another example, the approximate or precise determination of a top (i.e., end) portion of the villus may be determined by the processor. Specifically, villi may be annotated using a separate annotation layer. As illustrated in FIG. 3, a line segment V is drawn from the base Vb of the villus to the villus tip Vt. The base Vb is a relative point 0 and is identified by the presence of a crypt, the tip Vt is a relative point 1. Other annotated features may be analyzed based upon their relative position along this line segment V (i.e., the villus axis). These annotations may be performed manually by a user, via edge detection or like image processing techniques, or by machine learning.

In some embodiments, these statistical representations and aggregate properties may be global (i.e., mean MVL), may be local (i.e., MVL per villus), may be weighted based on a location of the MVL, and/or may be weighed based on image properties (i.e., whether a particular area of an image is more granular). For example, an average MVL determined in a first region may be weighted greater than an average MVL in a second region, to determine a weighted average of MVL across the first and second regions.

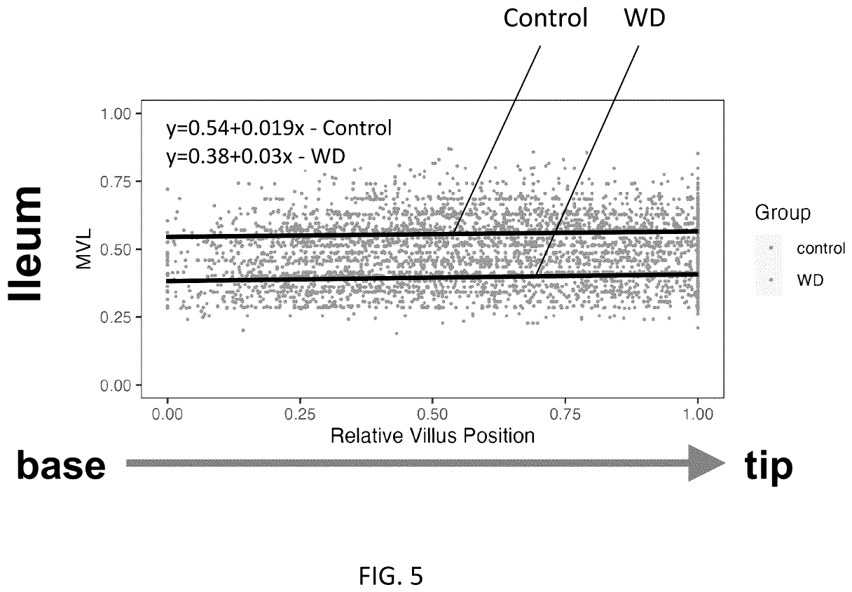

In some embodiments, a variation of MVL or an aggregate representation of MVL may be determined across a portion of the region or image. For example, as illustrated in FIG. 5, an average MVL may be determined as a function of position along a villi from the base to the tip; or average MVL of a villi may be determined as a function of location along the cephalo-caudal axis of the small intestine. Still further statistical parameters may be determined based on the aggregate parameter. For example, a slope of the MVL as a function of length may be determined. In other words, the rate of change of the MVL over a portion or all of a villi may be identified to determine how gradually (or abruptly) MVL changes along the length of the villi.

Intestinal epithelial cells (containing microvilli)_are elongated and tightly packed together. Nuclei are arranged next to one another in a nucleus-membrane-nucleus pattern along the villus creating a repeated light-dark-light grayscale pattern which can be detected using segmentation, edge detection, and similar techniques. Considering this, in some embodiments the processor may be further configured to identify individual cells along the villus, for example by detecting their nuclei. For each nucleus, the closest region of the microvillar brush is detected by the grayscale gradient relative to the nuclear axis and/or the villus axis and a corresponding MVL measurement is obtained. Each microvillus can then be associated to the closest corresponding nucleus measured perpendicularly from the microvillar brush.

Given this understanding, the association between microvilli and cells may be determined based on a distance matrix determined for each nucleus midpoint and every MVL line-segment midpoint. In some embodiments, the distance may be maxed at 160. Applying an optimization technique results in the optimal pairing of nuclei and MVL line segments, thereby minimizing the overall distance between the two objects. Prior to applying the optimization technique, each cell may be confirmed to have at least one microvillus associated to it. The grayscale gradient can then be determined along the determined line segments.

In one example, nuclei identification is achieved according to a machine learning system. In particular, the machine learning system may be generated by first annotating a set of nuclei on using polygons. An image may then be extracted from each polygon and used to train to the machine learning system (e.g., including a random forest classifier) on features extracted from the polygon images. New images may then be input to the machine learning system, which is trained to perform a pixel-level classification of the image, identifying each pixel as “nucleus” or “not nucleus.” These classifications may be used to find contours over the pixel-level classifications, convert the contours to polygons, and identify a polygon that overlaps with a selected point. In the event two nuclei overlap with one another, a watershed algorithm of a Sobel filter of the detected polygon may be used to sub-segment the polygon into two parts.

Associated statistical parameters may then be described at the cellular level, such as the rate of change of MVL, the cellular-level variation in MVL, and the like. In some embodiments, the statistical representations and/or aggregate properties can be compared across multiple regions. For example, an average MVL of a region including a lower third to a lower half of one or more villi may be compared to an average MVL of a region including an upper third to an upper half of one or more villi. This comparison may also be used to determine variation of MVL over a length of the villi.

In some embodiments, the determined MVL and related parameters may be compared across a time period and/or different subjects. For example, changes in MVL and related parameters may be determined between images taken from a subject at two different times. Comparison over time may be used to determine disease or health progression. Comparison made to different subjects (e.g., a normative database or subjects with known disease states) can be used to determine a disease state of the analyzed subject.

A diagnosis of the subject may then be determined based on any of the above-noted MVL and/or related parameters. For example, the processor may be configured to identify a disease state (e.g., inflammatory bowel disease, or the like) based on the MVL and related parameters. In one embodiment, decreased MVL in ileal biopsies may be associated with a diagnosis of Crohn's disease. Further, decreased MVL may be associated with poor response to some therapies.

Similarly, the absorption ability of the intestine (or other measure of the health of the intestine) may be quantified based on the determined MVL and related parameters, and this quantified intestinal health may be indicative of a diagnosis. This diagnosis may be further based on the metadata associated with the image of the subject. For example, microvillus blunting, as observed in patients with IBD and in preclinical animal models subjected to dietary manipulations, may be associated with not only decreased absorptive area, but also compositional changes in the enzymatic milieu required for optimal nutrient acquisition. In a further example, microvillus shortening is understood to be an adaptive response that may be associated with nutritional surplus/overload.

Still further, in response to the diagnosis, a treatment protocol may be recommended and output to a clinician based on clinical guidelines. Such a treatment protocol may also be based on results of another model that is trained on observational data and/or randomized control trial (RCT) data to predict disease progression based on the MVL. Still further, the treatment protocol may be based on the metadata associated with the image of the subject.

Following analysis, the system may output a result based on the analysis or the recommended treatment protocols. In one example, the output is provided to a display of the system. The result may comprise the determined MVL and related parameters, the determined disease state and/or change thereof, and/or a score of absorption ability and/or intestinal health. Further, the output may include a topographic map, heatmap, or the like of the MVL or related parameter. The topographic map may, for example, include an area of contours, slopes, and/or any dimensional parameters of any number of microvilli and/or villi. Accordingly, the MVL and/or related parameter may be shown as a two dimensional (2D) map. Such a map may be, for example, overlaid on the images and/or regions from which the MVL or related parameter was determined.

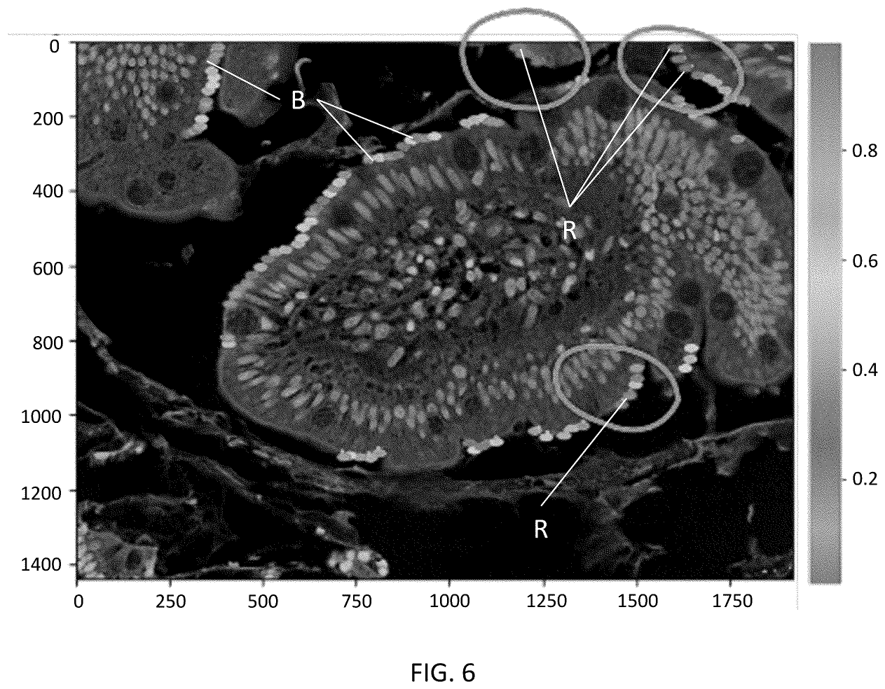

An example output is illustrated in FIG. 6. Therein, a spatial analysis of MVL variance is shown in a representative heatmap in which micro-regions with relative longer R and shorter B microvilli are colored differently. For example, the longer microvilli R may be indicated by red tones, while the shorter microvilli B may be indicated by bluet ones. Microvilli with lengths therebetween may indicated by other colors within the color spectrum.

As suggested, any of the above-described processing and analysis may performed by the processor (or a plurality of processors working cooperatively) of the system. For example, analysis of the segmented one or more regions of the image may be performed by a first processor, such that a second processor may be configured to receive results of the analysis and prepare an output. According to another example, each of the segmented one or more regions may be separately performed before being aggregated by or transmitted to analysis by one or more processors.

Further, as also suggested, any or all of the above analysis may be performed by one or more machine learning systems. For example, the image may be input to a learning model that is trained to segment the image, identify nuclei or cells, determine MVL and the like. A learning model may also or alternatively be trained to classify or predict a state and a degree of severity of a type of disease. In some embodiments, the system may be configured to achieve a desired level of confidence based on suggesting a number of images that are to be analyzed. Such learning model may include a deep learning model(s), convolutional neural networks (CNNs), random forest classifiers, and the like.

While various features are presented above, it should be understood that the features may be used singly or in any combination thereof. Further, it should be understood that variations and modifications may occur to those skilled in the art to which the claimed examples pertain.

What is claimed is:

1. A method comprising:
receiving a digitized pathology image of a subject;
segmenting the image to identify a region of interest that includes at least a portion of at least one villus;
identifying a pathological feature of the image within the region of interest;
determine a parameter pertaining to each of at least one instance of the identified pathological feature in the image;
determining an aggregate parameter based on the determined parameter; and
outputting a report of the determined aggregate parameter.

2. The method according to claim 1, wherein the pathological feature is a microvillus.

3. The method according to claim 1, wherein the pathological feature is a cell nucleus.

4. The method according to claim 1, wherein the pathological feature is a goblet cell.

5. The method according to claim 1, wherein the parameter is a microvillus length.

6. The method according to claim 1,
wherein the identified pathological feature is a villus,
wherein identifying at least one pathological feature comprises identifying contours corresponding to inner and outer edges of a brush border of at least one villi in the region of interest,
wherein determining the parameter comprises determining a distance between the contours, and
wherein the parameter is the distance between the contours.

7. The method according to claim 1,
wherein the identified pathological feature is a villus,
wherein identifying at least one pathological feature comprises:
identifying a contour corresponding to an edge of at least one villi in the region of interest; and
segmenting the contour;
wherein determining the parameter comprises, for each contour segment:
determining a direction toward an intracellular compartment of an epithelial cell corresponding to the segment; and
determining a distance between the segment and a pixel having a drop in intensity greater than a threshold along a path that extends normal to the segment in the determined direction, and
wherein the parameter corresponds to the determined distances.

8. The method according to claim 7, wherein the threshold is based on an average greatest pixel intensity drop across all of the segments.

9. The method according to claim 1, further comprising:
classifying or predicting a state or a degree of a disease of the subject based on the aggregate parameter.

10. The method according to claim 1, further comprising:
quantifying an intestinal health of the subject based on the aggregate parameter.

11. The method according to claim 1, further comprising:
identifying a treatment protocol for the subject based on the aggregate parameter.

12. The method according to claim 11, further comprising:
treating the subject based on the treatment protocol.

13. The method according to claim 1, wherein the outputted report comprises a heatmap of the determined parameter or the determined aggregate parameter overlaid on the digitized image.

14. The method according to claim 1, further comprising:
comparing the determined parameter or the determined aggregate parameter across a plurality of regions of interest or a plurality of digitized images.

15. The method according to claim 1, wherein the aggregate parameter is determined based on a comparison of the determined parameter across a plurality of the identified pathological features, a plurality of regions of interest, or a plurality of digitized images.

16. A system comprising:
at least one memory and processor configured to execute the method of claim 1.

17. The system according to claim 16, wherein the at least one processor comprises at least one trained machine learning system.
